(19) United States
(12) Patent Application Publication
(10) Pub. No.: US 2024/0084339 A1
(43) Pub. Date: Mar. 14, 2024

(54) CULTURE DEVICE AND USE THEREOF

(71) Applicant: KANEKA CORPORATION, Osaka-shi (JP)

(72) Inventors: Takafumi IGARI, Takasago-shi (JP); Takahisa KATO, Takasago-shi (JP); Masaru HIRANO, Takasago-shi (JP)

(73) Assignee: KANEKA CORPORATION, Osaka-shi (JP)

(21) Appl. No.: 18/263,063

(22) PCT Filed: Jan. 20, 2022
(86) PCT No.: PCT/JP2022/001928

§ 371 Date: Jul. 26, 2023

(30) Foreign Application Priority Data
Feb. 4, 2021 (JP) 2021-016726

(51) Int. Cl. C12P 7/625; C12M 1/06

(52) U.S. Cl. CPC C12P 7/625; C12M 27/02

(57) ABSTRACT

An object of the present invention is to provide a culture device capable of accurately detecting the gas-liquid interface of a culture solution in a fermenter. The above problem is solved by provided a culture device that includes: a liquid surface sensor for detecting a foam layer height from the bottom of the fermenter to the top of a foam layer; and a pressure sensor for detecting a liquid surface height from the bottom of the fermenter to a gas-liquid interface, the culture device having installed at least two pressure sensors below the gas-liquid interface, the at least two pressure sensors each being the pressure sensor.

### TECHNICAL FIELD

The present invention relates to a culture device and the use of the same.

### BACKGROUND ART

In the fed-batch culture for the production of a polyhydroxyalkanoate (which can hereinafter be referred to as “PHA”) the substrate of which is a fatty acid, there is the risk of a large amount of bubble holding in a culture solution. With a large amount of bubble holding, the apparent volume of the culture solution increases. This prevents efficient use of the volumetric capacity of the fermenter, and therefore leads to a production decrease. Further, when the bubbles and the solution become united, an oxygen transfer rate decreases, and therefore leads to a productivity decline. Thus, it is necessary to detect whether bubbles are held in the solution.

As a device for detecting the bubble holding, for example, disclosed in Patent Literature 1 is a bubbling phenomenon detecting device that includes: differential pressure-type level gauge for detecting a liquid level in a liquid tank having a reservoir section; and a capacitive level switch for detecting a foam level.

Further, Patent Literature 2 discloses a foam layer detecting device for detecting a foam layer generated on the surface of a foamable liquid in a tank through the combined use of a pressure-type liquid surface detecting sensor provided below the surface and a light measurement-type liquid level sensor provided above the surface.

### CITATION LIST

### Patent Literature

[Patent Literature 1]
Japanese Patent Application Publication, Tokukai, No. 2004-012226
[Patent Literature 2]
Japanese Utility Model Registration Application Publication, Jitsukaihei, No. 7-003703

### SUMMARY OF INVENTION

### Technical Problem

However, the devices disclosed in Patent Literatures 1 and 2 have only one pressure sensor installed in a liquid phase in the fermenter, and it can be therefore difficult to perform accurate foam-liquid surface management when the liquid density changes during culture. Thus, the devices are susceptible to improvement.

To address the above problem, an object of the present invention is to provide a culture device capable of accurately detecting the gas-liquid interface of a culture solution in a fermenter.

### Solution to Problem

As a result of conducting a diligent study to solve the above problem, the inventors of the present invention have become the first inventors to find that the combined use of at least two pressure sensors installed in a liquid phase and a liquid surface sensor allows accurate detection of the gas-liquid interface of a culture solution in a fermenter. Further, the inventors have also become the first inventors to find that using a culture device that includes the above sensors makes it possible to reduce the holding of bubbles in the culture solution, and therefore improve production per batch. Thus, the present invention has been completed.

Therefore, an aspect of the present invention is a culture device that includes a liquid surface sensor for detecting a foam layer height from the bottom of a fermenter to the top of a foam layer and a pressure sensor for detecting a liquid surface height from the bottom of the fermenter to a gas-liquid interface, the culture device having at least two pressure sensors installed below the gas-liquid interface, the at least two pressure sensors each being the pressure sensor.

Advantageous Effects of Invention

With an aspect of the present invention, it is possible to provide a culture device capable of accurately detecting the gas-liquid interface of a culture solution in a fermenter.

### BRIEF DESCRIPTION OF DRAWINGS

FIG. 1 is a schematic view of s culture device in accordance with an embodiment of the present invention.

### DESCRIPTION OF EMBODIMENTS

The following description will discuss an embodiment of the present invention in detail. The expression “A to B”, representing a numerical range, herein means “not less than A and not more than B” unless otherwise specified in this specification. All of the documents cited herein are incorporated herein by reference.

[1. Overview of Present Invention]

A culture device (hereinafter referred to as the “present culture device”) in accordance with an embodiment of the present invention includes a liquid surface sensor for detecting a foam layer height from the bottom of a fermenter to the top of a foam layer and a pressure sensor for detecting a liquid surface height from the bottom of the fermenter to a gas-liquid interface, the culture device having at least two pressure sensors installed below the gas-liquid interface, the at least two pressure sensors each being the pressure sensor.

As a result of the study conducted by the inventors of the present invention, it has been found that with the foam surface management used in, for example, beer production and carried out via, for example, the liquid surface sensor, there is the problem of being incapable of distinguishing between the surface of foam and the foam-liquid unified state. It has been found that, for example, with the techniques disclosed in Patent Literatures 1 and 2, in which there is only one pressure sensor installed in the liquid phase in a fermenter, it can be difficult to perform accurate foam-liquid surface management when the liquid density changes during culture.

As a result of diligently studying a culture device capable of accurately perform foam-liquid surface management in order to improve production efficiency in culture, the inventors of the present invention have successfully obtained the following findings.

It is possible to accurately detect the gas-liquid interface of a culture solution in a fermenter, by using at least two pressure sensors installed in a liquid phase and a liquid surface sensor in combination in a culture device.

It is possible to accurately detect the gas-liquid interface of a culture solution even in a case where the liquid density changes during culture, by using at least two pressure sensors installed in a liquid phase and a liquid surface sensor in combination in a culture device.

It is possible to reduce the holding of bubbles in a culture solution and thus improve production per batch, by using the above culture device.

The culture device having such a distinctive combined use of sensors and arrangement is an unprecedented device, and is a very excellent technique. Here is a detailed description of the configuration of the present production method.

[2. Culture Device]

(Culture Device)

The present culture device is characterized by including a liquid surface sensor for detecting a foam layer height from the bottom of a fermenter to the top of a foam layer and a pressure sensor for detecting a liquid surface height from the bottom of the fermenter to a gas-liquid interface, the culture device having at least two pressure sensors installed below the gas-liquid interface, the at least two pressure sensors each being the pressure sensor. The present culture device, which has the above configuration, is capable of accurately detecting the gas-liquid interface of a culture solution in a fermenter. Furthermore, even in a case where the liquid density changes during culture, it is possible to detect the gas-liquid interface of the culture solution, and thus reduce the holding of bubbles in the culture solution.

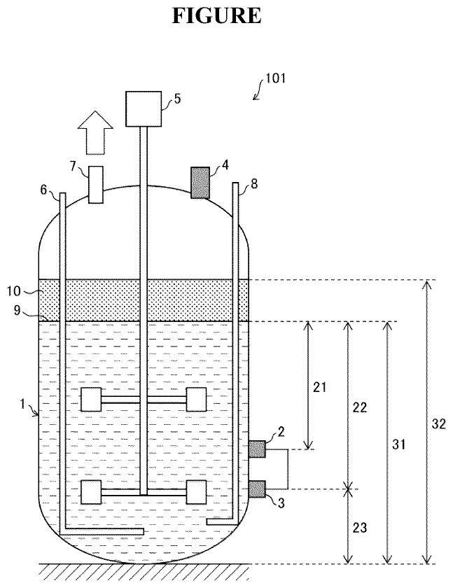

The present culture device will be described in detail with use of FIG. 1. Note that the present culture device is not limited to the culture device illustrated in FIG. 1. Further, although the following description is presented by taking the example in which at least two pressure sensors are installed below the gas-liquid interface, the present invention is not limited to this configuration, but at least three pressure sensors may be installed. In such a case, in which at least three pressure sensors are installed, it is only necessary to install at least two pressure sensors below the gas-liquid interface.

As illustrated in FIG. 1, the present culture device 101 includes: a pressure sensor (differential pressure-type liquid surface sensor) composed of an upper pressure sensor 2 and a lower pressure sensor 3; and a liquid surface sensor 4. A fermenter 1 contains a culture solution. The upper pressure sensor 2 and the lower pressure sensor 3 are installed below a gas-liquid interface 9 of the fermenter 1. Air is constantly supplied through an air supply pipe 6. The supplied air is dispersed in the culture solution by a stirrer 5. The air dispersed in the culture solution is then discharged through a discharge line 7. In addition, a carbon source is fed through a carbon source feed line 8. By changing a culture condition such as a carbon source addition rate, the amount of bubble holding in the culture solution changes, and the height of the gas-liquid interface 9 and the amount of formation of a foam layer 10 therefore change.

A liquid surface height 31 is detected by the pressure sensor (differential pressure-type liquid surface sensor). The liquid surface height 31 is intended to mean the distance from the bottom of the fermenter 1 to the gas-liquid interface 9. Specifically, the two pressure sensors (upper pressure sensor 2 and lower pressure sensor 3) are located below the gas-liquid interface 9, so that a difference is caused between the pressures detected by the respective pressure sensors. This allows the detection of the liquid surface height 31 based on the pressure difference. In addition, since the calculation of a liquid surface height is based on the pressure difference, it is possible to detect the liquid surface height 31 even in a case where the density of the culture solution changes during culture.

A foam layer height 32 is detected by the liquid surface sensor 4. The foam layer height 32 is the sum of the liquid surface height 31 and the foam layer 10. Therefore, the foam layer height 32 is intended to mean the distance from the bottom of the fermenter 1 to the top of the foam layer 10. By comparing the liquid surface height 31 measured via the pressure sensor (differential pressure-type liquid surface sensor) with the foam layer height 32 measured via the liquid surface sensor 4, it is possible to estimate the amount of formation of the foam layer 10. For example, when the value of the ratio of the liquid surface height 31 measured via the pressure sensor (differential pressure-type liquid surface sensor)/the foam layer height 32 measured via the liquid surface sensor 4 is closer to 1, it can be determined that the amount of formation of the foam layer 10 is smaller, and when the value of the ratio of the liquid surface height 31 measured via the pressure sensor (differential pressure-type liquid surface sensor)/the foam layer height 32 measured via the liquid surface sensor 4 is smaller than 1, it can be determined that the amount of formation of the foam layer 10 is greater. By changing, as appropriate, a culture condition according to such an amount of formation of the foam layer (e.g., the value of the ratio of the liquid surface height 31 measured via the pressure sensor (differential pressure-type liquid surface sensor)/the foam layer height 32 measured via the liquid surface sensor 4), it is possible to improve culture efficiency.

The liquid surface sensor 4 in the present culture device 101 is not particularly limited provided that the sensor is capable of detecting the foam layer height 32 from the bottom of the fermenter 1 to the top of the foam layer 10, but examples thereof include a laser liquid level gauge, a ultrasonic level gauge, a microwave radar-type level gauge, and a capacitive level gauge. The liquid surface sensor is preferably a laser liquid level gauge from the viewpoint of being capable of noncontact measurement and being inexpensive.

The pressure sensors 2 and 3 of the present culture device 101 is not particularly limited provided that the sensors are capable of detecting the liquid surface height 31 from the bottom of the fermenter 1 to the gas-liquid interface 9, but examples thereof include a differential pressure-type level sensor (DP cell). Two pressure sensors may be installed at the locations of the pressure sensors 2 and 3 in order for the pressure difference to be measured.

(Liquid Surface Height/Foam Layer Height)

According to an embodiment of the present invention, the present culture device 101 preferably includes an adjustment mechanism for adjusting a culture condition such that the numerical value of the ratio of the liquid surface height 31 measured via the pressure sensors 2 and 3/the foam layer height 32 measured via the liquid surface sensor 4 is within a certain range. With use of the adjustment mechanism, the culture condition is adjusted such that the numerical value of the ratio of the liquid surface height 31 measured via the pressure sensors/the foam layer height 32 measured via the liquid surface sensor 4 is preferably 0.85 to 0.99, more preferably 0.85 to 0.95, and even more preferably 0.85 to 0.92. When the numerical value is adjusted so as to be not more than 0.99, the foam layer 10 is present, and the gas holdup ratio (described later) is therefore not too high. When the numerical value is adjusted so as to be not less than 0.85, the culture efficiency improves.

The liquid surface height 31 can be measured via, for example, a differential pressure-type pressure sensor such as a DP cell. The differential pressure-type pressure sensor uses the difference between the pressures detected by the two pressure sensors 2 and 3 to measure the liquid surface height 31. The liquid surface height 31 can be calculated by summing up a distance 22 from the gas-liquid interface 9 to the lower pressure sensor 3 and a distance 23 from the lower pressure sensor 3 to the bottom of the fermenter 1. The distance 22 from the gas-liquid interface 9 to the lower pressure sensor 3 can be determined by Formula (1) below.

$$\\ \begin{matrix}L 2 = \frac{P 2}{P 2 - P 1} \times ( L 2 - L 1 ) & ( 1 )\end{matrix}$$

In Formula (1), P1 represents the pressure detected by the upper pressure sensor 2, P2 represents the pressure detected by the lower pressure sensor 3, L1 represents a distance 21 from the gas-liquid interface 9 to the upper pressure sensor 2, and L2 represents the distance 22 from the gas-liquid interface 9 to the lower pressure sensor 3. Note that the term (L2-L1) is not particularly limited provided that the distance causes a difference between the pressures detected by the respective pressure sensors, as described above.

(Culture Condition Adjustment Mechanism)

The culture condition adjustment mechanism in the present culture device 101 is capable of adjusting at least one selected from among, for example, a carbon source addition rate, a culture solution stirring power, a bubbling condition, and a stirring blade shape. That is, the adjustment mechanism is capable of adjusting the amount of formation of the foam layer 10 by controlling, for example, a carbon source addition rate, a culture solution stirring power, a bubbling condition, and/or a stirring blade shape, to adjust the ratio of the liquid surface height 31 measured via the pressure sensors 2 and 3/the foam layer height 32 measured via the liquid surface sensor 4 to an appropriate range.

According to an embodiment of the present invention, the adjustment of a culture condition by the adjustment mechanism is preferably carried out through at least one selected from the group consisting of a carbon source addition rate, a culture solution stirring power, and a bubbling condition, from the viewpoint of conveniently and efficiently changing the ratio of the liquid surface height 31 measured via the pressure sensors 2 and 3/the foam layer height 32 measured via the liquid surface sensor 4.

In a case where the adjustment of a culture condition by the adjustment mechanism is carried out through a carbon source addition rate, when the carbon source addition rate is increased, the amount of formation of the foam layer increases, and when the addition rate is reduced, the amount of formation of the foam layer 10 decreases. The carbon source addition rate (L/hr) with respect to the volumetric capacity (L) of the fermenter (hereinafter, also referred to as “addition rate/fermenter volumetric capacity” is, for example, 2.0 to 3.8 [1/hr], preferably 2.5 to 3.75 [1/hr], and more preferably 3.0 to 3.7 [1/hr]. When the carbon source addition rate is within the above range, it is possible to adjust the ratio of the liquid surface height 31 measured via the pressure sensors 2 and 3/the foam layer height 32 measured via the liquid surface sensor 4 to an appropriate range. As a result, it is possible to enhance production per batch. The adjustment mechanism for adjusting the carbon source addition rate can be, for example, the carbon source feed line 8 illustrated in FIG. 1.

The carbon source is not particularly limited, but is preferably a surface-active carbon source from the viewpoint of the dispersibility in the culture solution. Examples of the surface-active carbon source include lipids derived from plant oil, glycerin, and polyhydric alcohols. In particular, lipids derived from plant oil are preferable from the viewpoint of the property of being utilized by fungi. The plant oil is not particularly limited, but examples thereof include palm oil, olive oil, corn oil, canola oil, coconut oil, soybean oil, wheat malt oil, jojoba oil, sunflower oil, sesame, peanuts, cotton seed, safflower, soybeans, rapeseeds, almonds, beechmast, cashews, hazelnuts, macadamias, mongongo nuts, pecans, pine nuts, pistachios, walnut, grapefruit seeds, lemon, orange, bitter melon, gourds, buffalo gourd, butternut seeds, egusi seeds, pumpkin seeds, watermelon seeds, acai seeds, blackseed, blackcurrant seeds, borage seeds, evening primrose, flax, eucalyptus, amaranth, apricot, apple seeds, argan, avocado, babassu, coriander seeds, grape seeds, mustard, poppy seeds, rice bran, castor-oil plant, and a combination thereof. In particular, palm oil is preferable from the viewpoint of availability.

In a case where the adjustment of a culture condition by the adjustment mechanism is carried out through the culture solution stirring power, when the culture solution stirring power is increased, the amount of formation of the foam layer 10 increases, and when the stirring power is reduced, the amount of formation of the foam layer 10 decreases. The stirring power per unit volume of the culture solution is, for example, 1.5 kw/m³ to 4.0 kw/m³, preferably 1.5 kw/m³ to 3.5 kw/m³, and more preferably 2.0 kw/m³ to 3.0 kw/m³. When the culture solution stirring power is within the above range, it is possible to adjust the ratio of the liquid surface height 31 measured via the pressure sensors 2 and 3/the foam layer height 32 measured via the liquid surface sensor 4 to an appropriate range. As a result, it is possible to enhance production per batch. The adjustment mechanism for adjusting the culture solution stirring power can be, for example, the stirrer 5 illustrated in FIG. 1.

In a case where the adjustment of a culture condition by the adjustment mechanism is carried out through the bubbling condition, when the amount of bubbling is increased, the amount of formation of the foam layer 10 increases, and when the amount of bubbling is reduced, the amount of formation of the foam layer 10 decreases. The bubbling condition is, for example, 0.2 vvm to 2.0 vvm, preferably 0.4 vvm to 1.5 vvm, and more preferably 0.6 vvm to 1.2 vvm. When the bubbling condition is within the above range, it is possible to adjust the ratio of the liquid surface height 31 measured via the pressure sensors 2 and 3/the foam layer height 32 measured via the liquid surface sensor 4 to an appropriate range. As a result, it is possible to enhance production per batch. The adjustment mechanism for adjusting the bubbling condition can be, for example, the air supply pipe 6 illustrated in FIG. 1.

(Gas Holdup Ratio)

According to an embodiment of the present invention, an aeration stir is carried out in culture in the present culture device 101. The aeration stir causes the culture solution to hold air, and gas holdup thus occurs.

As used herein, the “gas holdup ratio” is intended to mean the proportion of the volume of bubbles to the entire volume of the culture solution in which the gas holdup occurs. When the gas holdup ratio is high, the volumetric capacity of the fermenter cannot be efficiently used. This reduces culture efficiency. However, also when the gas holdup ratio is low, air is less soluble in the culture solution, and culture efficiency therefore reduced. Accordingly, the gas holdup ratio is preferably controlled so as to fall within a certain range.

According to an embodiment of the present invention, the gas holdup ratio (c) is, for example, 0.20 to 0.32, preferably 0.23 to 0.30, and more preferably 0.26 to 0.29. When the gas holdup ratio is not less than 0.20, air is more soluble in the culture solution, and when the gas holdup ratio is 0.32, it is possible to efficiently use the volumetric capacity of the fermenter 1, and therefore possible to increase productivity per batch. The gas holdup ratio ε is defined by Formula (2) below.

$$\\ \begin{matrix}ε = \frac{{V}_{f} - {V}_{0}}{{V}_{f}} & ( 2 )\end{matrix}$$

In Formula (2), V_{f} represents the volume of the culture solution at the time of occurrence of gas holdup, and Vo represents the amount of the culture solution put in the fermenter 1.

The gas holdup ratio is controlled via the air supply pipe 6 illustrated in FIG. 1.

(Others)

The volumetric capacity of the fermenter 1 of the present culture device 101 and the ratio of the height of the fermenter 1/the diameter of the fermenter 1 are not particularly limited, provided that the volumetric capacity and the ratio are great enough to enable the pressure sensors 2 and 3 to be installed so as to be separated from each other by the distance enough to generate a difference between the respective pressures detected by the upper pressure sensor 2 and the lower pressure sensor 3.

The volumetric capacity of the fermenter 1 of the present culture device 101 is, for example, 0.4 m³ to 350 m³, preferably 1 m³ to 330 m³, and more preferably 2 m³ to 300 m³.

According to an embodiment of the present invention, in the present culture device 101, the ratio of the height of the fermenter 1/the diameter of the fermenter 1 is, for example, 1.5 to 3.0, preferably 1.7 to 2.7, and more preferably 1.9 to 2.5.

According to an embodiment of the present invention, it is preferable that in the present culture device 101, the fermenter 1 have a volumetric capacity of 0.4 m³ to 350 m³ and the ratio of the height of the fermenter 1/the diameter of the fermenter 1 be 1.5 to 3.0.

The fermenter 1 of the present culture device 101 is not particularly limited, but is preferably an SUS (stainless steel) container from the viewpoint of allowing for greater volume.

(Microorganism)

With the present culture device 101 described above, it is possible to adjust the foam layer 10 and the gas holdup ratio in the culture solution, to culture microorganisms in the culture solution.

The microorganisms are not particularly limited, but examples thereof include microorganisms capable of producing biodegradable plastic, which has little adverse effect on the ecosystem and is thus environmentally friendly. In particular, microorganisms which use natural organic acids and oils derived from plants as carbon sources to produce PHAs and which accumulate, in the cells thereof, the PHAs, which are energy accumulating substances are preferable.

The PHA is a general term for polymers the monomer unit of which is a 3-hydroxyalkanoate. The 3-hydroxyalkanoate of the PHA is not particularly limited, but examples thereof include 3-hydroxypropionate, 3-hydroxybutyrate, 3-hydroxyvalerate, 3-hydroxyhexanoate, 3-hydroxyheptanoate, and 3-hydroxyoctanoate. The PHA may be a homopolymer the monomer unit of which is one type of 3-hydroxyalkanoate, or may be a copolymer the monomer unit of which is at least two types of 3-hydroxyalkanoates. Examples of the copolymer include a copolymer of 3-hydroxybutyrate (3HB) and another type of 3-hydroxyalkanoate and a copolymer of 3-hydroxyalkanoates in which at least 3-hydroxyhexanoate (3HH) is contained as the monomer unit. As the PHA, specifically, the following are preferable from the viewpoint of being industrially easily produced: poly(3-hydroxybutyrate) (PHB), poly(3-hydroxybutyrate-co-3-hydroxyhexanoate) (PHBH), poly(3-hydroxybutyrate-co-3-hydroxyvalerate-co-3-hydroxyhexanoate), poly(3-hydroxybutyrate-co-3-hydroxyhexanoate), poly(3-hydroxybutyrate-co-4-hydroxybutyrate), poly(3-hydroxybutyrate-co-3-hydroxyoctanoate), poly(3-hydroxybutyrate-co-3-hydroxyoctadecanoate), and the like.

The microorganisms used for the production of the PHA is not particularly limited provided that the microorganisms are capable of producing PHAs. The microorganisms that can be used are, for example, a microorganism isolated from nature, a microorganism deposited with a depositary institution (e.g., IFO and ATCC) for strains, a genetically-engineered microorganism such as a mutant or a transformant that can be prepared from the aforementioned microorganisms.

Specific examples of the microorganisms include microorganisms of: genus Cupriavidus such as Cupriavidus necator, genus Alcaligenes such as Alcaligenes latas; genus Pseudomonas such as Pseudomonas putida, Pseudomonas fluorescens, Pseudomonas aeruginosa, Pseudomonas resinovorans, or Pseudomonas oleovorans; genus Bacillus such as Bacillus megaterium; genus Azotobacter; genus Nocardia; genus Aeromonas such as Aeromonas caviae or Aeromonas hydrophila; genus Ralstonia; genus Wautersia; and genus Comamonas (Microbiological Reviews, 54(4), 450-472 (1990)).

In addition to the microorganisms above, biological tissue having artificially been modified so as to produce PHAs, by using a genetic engineering procedure to introduce a PHA synthesis enzyme gene or the like, can also be used. For example, in addition to the microorganisms of, for example, genera Cupriavidus, Alcaligenes, Pseudomonas, Bacillus, Azotobacter, Nocardia, Aeromonas, Ralstonia, Wautersia, and Comamonas, Gram-negative bacteria such as bacteria of genus Escherichia, Gram-positive bacteria such as bacteria of genus Bacillus, yeasts such as yeasts of genera Saccharomyces, Yarrowia, and Candida, and the like can be appropriately used to obtain the biological tissue having artificially been modified so as to produce PHAs.

Culture of the microorganisms can be carried out by culture methods similar to those commonly used for culturing the respective microorganisms.

From the microorganisms having been cultured by the above culture methods, it is possible to recover PHAs with use of well-known methods. For example, the following method can be used for the recovery. After the completion of the culture, microbial cells are separated from a culture solution by a centrifuge or the like, and the microbial cells are washed with distilled water and methanol or the like, and dried. From these dried microbial cells, a solution containing PHAs are extracted with use of an organic solvent such as chloroform. From this solution containing the PHAs, the microbial cell component is removed by filtering, and methanol or a poor solvent such as hexane is added to the filtrate, so that the PHAs precipitate. Further, the supernatant liquid is removed by filtering and centrifugation and is dried, so that the PHAs are recovered. Note that the poor solvent denotes a solvent having a lower degree of solubility than a product has.

The culture solution used for the culture of the microorganisms is not particularly limited, but a known culture solution can be used.

[3. Culture Method]

According to an embodiment of the present invention, a culture method (hereinafter referred to as the “present culture method”) is provided, the culture device including using the present culture device to culture microorganisms. With the present culture method, in which the present culture device is used, it is possible to accurately detect the gas-liquid interface of a culture solution in a fermenter, and reduce the holding of bubbles in the culture solution to increase productivity.

According to an embodiment of the present invention, the present culture method preferably includes the following Steps (a) and (b).
Step (a): A step of using the present culture device to measure the liquid surface height and the foam layer height.
Step (b): A step of adjusting a culture condition such that the ratio of the liquid surface height measured via the pressure sensors/the foam layer height measured via the liquid surface sensor is 0.85 to 0.99.

In the present embodiment, on the basis of a liquid surface height and a foam layer height measured with use of the present culture device, the culture condition is adjusted such that the liquid surface height and the foam layer height are in certain ranges.

In Step (a), the measurement of the liquid surface height and the foam layer height are carried out by the method described in [2. Culture device]. In Step (b), the adjustment of the ratio of the liquid surface height measured via the pressure sensors/the foam layer height measured via the liquid surface sensor is carried out by the method described in [2. Culture device].

According to an embodiment of the present invention, the present culture method preferably further includes the following Step (c).
Step (c): A step of controlling a gas holdup ratio in the culture solution such that the gas holdup ratio is 0.20 to 0.32.
   With Step (c), it is possible to efficiently use the volumetric capacity of the fermenter and thus increase productivity per batch.

Note that the terms such as “microorganisms”, “the adjustment of a culture condition”, “the volumetric capacity of a fermenter”, and “the height of a fermenter/the diameter of a fermenter” described in [2. Culture device] are employed in the present culture method.

[4. Method for Producing Polyhydroxyalkanoate]

According to an embodiment of the present invention, a PHA production method (hereinafter referred to as the “present production method”) is provided, the PHA production method including a step of using the present culture device to culture microorganisms or a step which is the present culture method. With the present production method, in which the present culture device or the present culture method is used, it is possible to accurately detect the gas-liquid interface of a culture solution in a fermenter, and reduce the holding of bubbles in the culture solution to increase productivity.

According to an embodiment of the present invention, the present production method can include Steps (a) and (b) or Steps (a) to (c) described in [3. Culture method].

According to an embodiment of the present invention, the microorganisms are preferably cultured in a culture solution that contains a surface-active carbon source. This advantageously provides an increase in polyhydroxyalkanoate productivity.

According to an embodiment of the present invention, the ratio of PHA weight/the volumetric capacity of the fermenter in the present production method is preferably 290 g/L, and more preferably 300 g/L. The ratio of PHA weight/the volumetric capacity of the fermenter is an index that indicates the productivity in producing the target substance.

According to an embodiment of the present invention, the present production method preferably includes, in addition to the step of culturing microorganisms, the optional steps below that follow the step of culturing microorganisms.
A step of inactivating the microorganisms
A step of disrupting the inactivated microorganisms
A step of separating a PHA from the disruption liquid obtained by the disruption and condensing the PHA
A step of drying the PHA aqueous suspension obtained by the condensation.

Each of these steps is carried out by any known method.

Note that the terms such as “microorganisms”, “PHA”, “culture”, “carbon source”, and “surface-active carbon source” described in [2. Culture device] are employed in the present production method.

The present invention is not limited to the above embodiments, but can be altered by a skilled person in the art within the scope of the claims. The present invention also encompasses, in its technical scope, any embodiment derived by combining technical means disclosed in differing embodiments.

Specifically, the present invention encompass the following embodiments.

<1> A culture device including: a liquid surface sensor for detecting a foam layer height from a bottom of a fermenter to a top of a foam layer; and a pressure sensor for detecting a liquid surface height from the bottom of the fermenter to a gas-liquid interface, the culture device having at least two pressure sensors installed below the gas-liquid interface, the at least two pressure sensors each being the pressure sensor.

<2> The culture device described in <1>, further including an adjustment mechanism for adjusting a culture condition such that a ratio of the liquid surface height measured via the at least two pressure sensors/the foam layer height measured via the liquid surface sensor is 0.85 to 0.99.

<3> The culture device described in <2>, in which the adjustment mechanism for adjusting the culture condition is capable of adjusting at least one selected from the group consisting of a carbon source addition rate, a culture solution stirring power, and a bubbling condition.

<4> The culture device described in any one of <1> to <3>, in which a volumetric capacity of the fermenter is 0.4 m³ to 350 m³, and a ratio of a height of the fermenter/a diameter of the fermenter is 1.5 to 3.0.

<5> A culture method including using the culture device described in <1> to culture microorganisms.

<6> The culture method described in <5>, including the steps of: (a) using the culture device described in <1> to measure the liquid surface height and the foam layer height; and (b) adjusting the culture condition such that the ratio of the liquid surface height measured via the at least two pressure sensors/the foam layer height measured via the liquid surface sensor is 0.85 to 0.99.

<7> The culture method described in <6>, in which adjusting the culture condition is adjusting at least one selected from the group consisting of a carbon source addition rate, a culture solution stirring power, and a bubbling condition.

<8> The culture method described in <7>, in which the carbon source addition rate is 2.0 [1/hr] to 3.8 [1/hr].

<9> The culture method described in <7>, in which the culture solution stirring power per unit volume of the culture solution is 1.5 kw/m³ to 4.0 kw/m³.

<10> The culture method described in <7>, in which the bubbling condition is 0.2 vvm to 2.0 vvm.

<11> The culture method described in any one of <5> to <10>, further including the step of (c) controlling a gas holdup ratio in the culture solution such that the gas holdup ratio is 0.20 to 0.32.

<12> A method for producing a polyhydroxyalkanoate, the method including the step of using the culture device described in any one of <1> to <4> to culture microorganisms, or a step which is the culture method described in any one of <5> to <11>.

<13> The method described in <12>, in which the microorganisms are cultured in a culture solution which contains a surface-active carbon source.

<14> The method described in <13>, in which the carbon source is a lipid derived from plant oil.

### EXAMPLES

The present invention will be described in detail below on the basis of an example. However, the present invention is not limited to this example.

[Measurement and Evaluation Method]

The measurements and evaluations in Example and Comparative Example were carried out by the following methods.

(Measurement of Liquid Surface Height)

The liquid surface height was measured (calculated) via a liquid surface differential pressure gauge. In brief, the liquid surface height was measured (calculated) with use of a difference between the respective pressures detected by two pressure sensors of the liquid surface differential pressure gauge. The distance (L2) from the gas-liquid interface to the lower pressure sensor was determined by Formula (1) below.

$$\\ \begin{matrix}L 2 = \frac{P 2}{P 2 - P 1} \times ( L 2 - L 1 ) & ( 1 )\end{matrix}$$

(In Formula (1), P1 represents the pressure detected by the upper pressure sensor, P2 represents the pressure detected by the lower pressure sensor, L1 represents a distance from the gas-liquid interface to the upper pressure sensor, and L2 represents the distance from the gas-liquid interface to the lower pressure sensor.

(Density of Culture Solution)

The density of the culture solution was calculated by dividing the liquid surface height (the volume of a liquid in the fermenter) measured via the liquid surface differential pressure gauge by the weight of the culture solution put in the fermenter.

### Production Example 1

In the culture production, KNK-631 strain (see Japanese Patent Application Publication, Tokukai, No. 2013-009627 and International Publication No. 2016/114128) is used to carry out mother culture and the subsequent preculture, to recover microbial cells.

(Culture Media)

The following culture media were used for mother culture, preculture, and main culture (described later).

<Culture Medium for Mother Culture>

The composition of the culture medium for mother culture was as follows: 1 w/v % of Meat-extract, 1 w/v % of Bacto-Tryptone, 0.2 w/v % of Yeast-extract, 0.9 w/v % of Na₂HPO₄·12H₂O, and 0.15 w/v % of KH₂PO₄. The pH of the culture medium was 6.8.

<Culture Medium for Preculture>

The composition of the culture medium for preculture was as follows: 1.1 w/v % of Na₂HPO₄·12H₂O, 0.19 w/v % of KH₂PO₄, 1.29 w/v % of (NH₄)₂SO₄, 0.1 w/v % of MgSO₄·7H₂O, and 0.5 v/v % of a trace metal salt solution (a solution in which 1.6 w/v % of FeCl₃·6H₂O, 1 w/v % of CaCl₂·2H₂O, 0.02 w/v % of CoCl₂·6H₂O, 0.016 w/v % of CuSO₄·5H₂O, and 0.012 w/v % of NiCl₂·6H₂O were dissolved in 0.1 N hydrochloric acid). As the carbon source, palm oil was collectively added in a manner that the concentration of the palm oil is 10 g/L.

<Culture Medium for Main Culture>

The composition of the main culture was as follows: 0.385 w/v % of Na₂HPO₄·12H₂O, 0.067 w/v % of KH₂PO₄, 0.291 w/v % of (NH₄)₂SO₄, 0.1 w/v % of MgSO₄·7H₂O, 0.5 v/v % of a trace metal salt solution (a solution in which 1.6 w/v % of FeCl₃·6H₂O, 1 w/v % of CaCl₂·2H₂O, 0.02 w/v % of CoCl₂·6H₂O, 0.016 w/v % of CuSO₄·5H₂O, and 0.012 w/v % of NiCl₂·6H₂O were dissolved in 0.1 N hydrochloric acid), and 0.05 w/v % of BIOSPUREX200K (defoaming agent manufactured by Cognis Japan Ltd.).

(Culture of Mother)

First, a glycerol stock of a KNK-631 strain was inoculated into the culture medium for the mother, and cultured at 30° C. for 24 hours, so that a mother culture solution was obtained.

(Preculture)

Into a container in which the culture medium for preculture was put, 1.0 v/v % of the mother culture solution obtained was inoculated. The culture was carried out for 24 hours while the culture temperature was controlled to be 30° C. and the pH was controlled to be 6.5. For the pH control, 14% aqueous ammonium hydroxide solution was used.

### Example 1

As the fermenter, a fermenter that is made of SUS and that has a volumetric capacity of 5.0 m³ and has the ratio of the height of the fermenter/the diameter of the fermenter is 2.5 was used. In the fermenter, a liquid surface differential pressure gauge (DP cell manufactured by Yokogawa Electric Corporation) was installed as the pressure sensor. The distance between the upper pressure sensor and the lower pressure sensor was set to 88 cm. In addition, as the liquid surface sensor, a radio wave-type liquid surface sensor (manufactured by Endress+Hauser) was installed at the top portion of the fermenter. Into the fermenter in which the culture medium for the main culture was put, the preculture solution obtained in the Production Example was inoculated such that the concentration of the preculture solution was 5.0 v/v %. The culture conditions were such that the culture temperature was 34° C., the stirring power was 2.5 kw/m³, and the ratio of an aeration amount/an initial liquid volume was 0.8 vvm. The pH was controlled to be 6.5. For the pH control, 14% aqueous ammonium hydroxide solution was used. Palm oil, which was the carbon source, was added such that the ratio of the addition rate/the volumetric capacity of the fermenter was 3.69×10⁻³ [1/hr].

After a lapse of 40 hours, the ratio (i.e., liquid surface height) of the volume/the volumetric capacity of the fermenter, the ratio being detected by the liquid surface differential pressure gauge, indicated 0.81, and the ratio (i.e., foam layer height) of the volume/the volumetric capacity of the fermenter, the ratio being detected by the radio wave-type liquid surface sensor, indicated 0.89 (the ratio of the liquid surface height/the foam layer height was 0.91). Thus, it was found that the foam layer was generated on the top of the surface of the culture solution, and the liquid surface differential pressure gauge was capable of detecting the surface of the liquid, and the radio wave-type liquid surface sensor was capable of detecting the surface of the foam layer. Further, the gas holdup ratio was 0.27.

At the completion of the culture that had been carried out for not less than 48 hours, the ratio of the final volume of the culture solution/the volumetric capacity of the fermenter, the ratio being detected by the liquid surface differential pressure gauge, was 0.87. The ratio of the weight of the PHA produced in the culture/the volumetric capacity of the fermenter was 303 g/L.

### Comparative Example 1

Culture was carried out in the same manner as in Example 1 except that palm oil, which was the carbon source, was added such that the ratio of the addition rate/the volumetric capacity of the fermenter was 3.84×10⁻³ [1/hr]. After a lapse of 40 hours, the ratio (i.e., liquid surface height) of the volume/the volumetric capacity of the fermenter, the ratio being detected by the liquid surface differential pressure gauge, indicated 0.82, and the ratio (i.e., foam layer height) of the volume/the volumetric capacity of the fermenter, the ratio being detected by the radio wave-type liquid surface sensor, also indicated 0.82 (the ratio of the liquid surface height/the foam layer height was 1.00). Thus, it was found that no foam layer was formed on the top of the culture solution, and the bubbles were completely held in the culture solution. Further, the gas holdup ratio was 0.33.

At the completion of the culture that had been carried out for not less than 48 hours, the ratio of the final volume of the culture solution/the volumetric capacity of the fermenter, the ratio being detected by the liquid surface differential pressure gauge, was 1.00. The ratio of the weight of the PHA produced in the culture/the volumetric capacity of the fermenter was 288 g/L.

[Results]

From the above, it has been found that it is possible to accurately detect the gas-liquid interface with use of a culture device that includes a liquid surface sensor for detecting a foam layer height on the top of a culture solution and a pressure sensor for detecting the gas-liquid interface of the culture solution, the culture device having two pressure sensors installed below the gas-liquid interface of the culture solution, the two pressure sensors each being the pressure sensor.

Further, by comparing Example with Comparative Example, it has been found that reducing the carbon source addition rate allows a reduction in the gas holdup in the culture solution. As a result, it was possible to efficiently use the volume of the fermenter, and thus increase the PHA production per batch.

Furthermore, as illustrated in Table 1, it has been found that in both of Example and Comparative Example, even when the density of the culture solution in the fermenter changes, it is possible to detect the gas-liquid interface of the culture solution.

TABLE 1

| | Example 1 | | Comparative Example 1 | |
|---|---|---|---|---|
| | | Density of | | Density of |
| Culture | Liquid surface | culture | Liquid surface | culture |
| time | height/Foam | solution | height/Foam | solution |
| [hr] | layer height | [g/mL] | layer height | [g/mL] |
| 8 | — | 0.74 | — | 0.81 |
| 24 | — | 0.75 | 0.96 | 0.68 |
| 32 | 0.91 | 0.79 | 1.04 | 0.69 |
| 40 | 0.90 | 0.75 | 1.00 | 0.75 |

### INDUSTRIAL APPLICABILITY

The present invention allows accurate detection of the gas-liquid interface of a culture solution in a fermenter. Thus, the present invention can be suitably used in a culture device for culturing, for example, microorganisms, and in any other field.

### REFERENCE SIGNS LIST

1: Fermenter
2: Upper pressure sensor
3: Lower pressure sensor
4: Liquid surface sensor
5: Stirrer
6: Air supply pipe
7: Discharge line
8: Carbon source feed line
9: Gas-liquid interface
10: Foam layer
21: Distance from gas-liquid interface to upper pressure sensor
22: Distance from gas-liquid interface to lower pressure sensor
23: Distance from lower pressure sensor to bottom of fermenter
31: Liquid surface height
32: Foam layer height
101: Culture device

1. A culture device comprising:
a liquid surface sensor for detecting a foam layer height from a bottom of a fermenter to a top of a foam layer; and
a pressure sensor for detecting a liquid surface height from the bottom of the fermenter to a gas-liquid interface,
the culture device having at least two pressure sensors installed below the gas-liquid interface, the at least two pressure sensors each being the pressure sensor,
the at least two pressure sensors comprising an upper pressure sensor and a lower pressure sensor, and
the at least two pressure sensors detecting the liquid surface height in accordance with a difference in pressure between the upper pressure sensor and the lower pressure sensor.

2. The culture device according to claim 1, further comprising:
an adjustment mechanism for adjusting a culture condition such that a ratio of the liquid surface height measured via the at least two pressure sensors/the foam layer height measured via the liquid surface sensor is from 0.85 to 0.99.

3. The culture device according to claim 2, wherein the adjustment mechanism for adjusting the culture condition is capable of adjusting at least one selected from the group consisting of a carbon source addition rate, a culture solution stirring power, and a bubbling condition.

4. The culture device according to claim 1, wherein a volumetric capacity of the fermenter is from 0.4 m³ to 350 m³, and a ratio of a height of the fermenter/a diameter of the fermenter is from 1.5 to 3.0.

5. A culture method comprising:
culturing microorganisms in the culture device of claim 1.

6. The culture method according to claim 5, comprising:
(a) measuring the liquid surface height and the foam layer height using the culture device of claim 1; and
(b) adjusting a culture condition such that a ratio of the liquid surface height measured via the at least two pressure sensors/the foam layer height measured via the liquid surface sensor is from 0.85 to 0.99.

7. The culture method according to claim 6, wherein adjusting the culture condition is adjusting at least one selected from the group consisting of a carbon source addition rate, a culture solution stirring power, and a bubbling condition.

8. The culture method according to claim 7, wherein the carbon source addition rate is from 2.0 [1/hr] to 3.8 [1/hr].

9. The culture method according to claim 7, wherein the culture solution stirring power per unit volume of the culture solution is from 1.5 kw/m³ to 4.0 kw/m³.

10. The culture method according to claim 7, wherein the bubbling condition is from 0.2 vvm to 2.0 vvm.

11. The culture method according to claim 5, further comprising:
(c) controlling a gas holdup ratio in the culture solution such that the gas holdup ratio is from 0.20 to 0.32.

12. A method for producing a polyhydroxyalkanoate, the method comprising:
culturing microorganisms in the culture device of claim 1.

13. The method according to claim 12, wherein the microorganisms are cultured in a culture solution comprising a surface-active carbon source.

14. The method according to claim 13, wherein the carbon source is a lipid derived from plant oil.

15. The culture device according to claim 1, wherein a volumetric capacity of the fermenter is from 0.4 m³ to 350 m³.

16. The culture device according to claim 1, wherein a ratio of a height of the fermenter/a diameter of the fermenter is from 1.5 to 3.0.

17. A method for producing a polyhydroxyalkanoate, the method comprising:
culturing microorganisms in the culture method of claim 6.

18. The method according to claim 17, wherein adjusting the culture condition is adjusting at least one selected from the group consisting of a carbon source addition rate, a culture solution stirring power, and a bubbling condition.

19. A method for producing a polyhydroxyalkanoate, the method comprising:
culturing microorganisms in the culture method of claim 9.

20. A method for producing a polyhydroxyalkanoate, the method comprising:
culturing microorganisms in the culture method of claim 11.
